(19) United States
(12) Patent Application Publication
(10) Pub. No.: US 2024/0265166 A1
(43) Pub. Date: Aug. 8, 2024

(54) GRATING-BASED X-RAY PHASE CONTRAST IMAGING SYSTEM SIMULATION

(71) Applicant: GE Precision Healthcare LLC, Wauwatosa, WI (US)

(72) Inventor: Uwe Wiedmann, Clifton Park, NY (US)

(21) Appl. No.: 18/163,510

(22) Filed: Feb. 2, 2023

(51) Int. Cl. G06F 30/20

(52) U.S. Cl. CPC G06F 30/20; A61B 6/583

(57) ABSTRACT

Computer processing techniques are described for simulating grating-based X-ray phase contrast imaging systems. According to an example, a system comprises a memory that stores computer-executable components and a processor that executes the computer-executable components stored in the memory. The computer-executable components comprise a simulation component that simulates performance of an X-ray phase contrast imaging system using a simulation model, wherein the simulation model comprises a decomposition component that decomposes a virtual object into different sub-objects associated with different physical properties, and a projector component that separately models different changes to a baseline interference pattern received at a detector of the X-ray phase contrast imaging system respectively attributed to the different sub-objects in association with simulated projection of an X-ray beam through the different sub-objects.

### TECHNICAL FIELD

This application relates to medical imaging systems and more particularly to computer processing techniques for simulating grating-based X-ray phase contrast imaging systems.

### BACKGROUND

X-ray-based imaging modalities including mammography and computed tomography (CT) are widely used in cancer screening, diagnosis, staging, treatment planning, and therapy response monitoring. Over the past few decades, improvements to these modalities have resulted in substantially improved efficacy and efficiency, and substantially reduced radiation dose and cost. However, such improvements have evolved more slowly than would be ideal because lengthy preclinical and clinical evaluation is required. In many cases, new ideas cannot be evaluated due to the high cost of fabricating and testing prototypes.

To accelerate development of new and improved imaging technologies, computer simulation tools have been proposed that provide for conducting imaging trials efficiently and realistically in a simulated environment. An X-ray-based cancer imaging simulation toolkit (XCIST) project supported by the National Cancer Institute of the National Institutes of Health has devoted significant research in developing a simulation tool for accurate simulation of the X-ray and CT imaging process, including state-of-the-art CT reconstruction algorithms, detailed and realistic digital representations of patients (“phantoms”) with and without realistic tumors, and a dose estimation tool. However, current computer simulation tools for accurately and efficiently performing X-ray phase contrast simulations, particularly as extended to CT, have yet to be realized.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products are described that facilitate simulating grating-based X-ray phase contrast imaging systems.

According to an embodiment, a system is provided that comprises a memory that stores computer-executable components, and a processor that executes the computer-executable components stored in the memory. The computer-executable components comprise a simulation component that simulates performance of an X-ray phase contrast imaging system using a simulation model, the simulation model comprising: a decomposition component that decomposes a virtual object into different sub-objects associated with different physical properties; and a projector component that separately models different changes to a baseline interference pattern received at a detector of the X-ray phase contrast imaging system respectively attributed to the different sub-objects in association with simulated projection of an X-ray beam through the different sub-objects. The different sub-objects comprise an absorption object, a phase object and a small angle scattering object. In this regard, the different changes comprise an absorption change (e.g., an amount decrease in X-ray photon absorption at respective detector elements/pixels) attributed to the absorption object, a phase change (e.g., a shift in the interference pattern at respective detector elements/pixels) attributed to the phase object, and a dark field change (e.g., a decrease in visibility and/or shape of the interference pattern at respective detector elements/pixels) attributed to the small-angle scattering object. The simulation model further comprises a detector component that combines the different changes together to generate an aggregated interference pattern for the virtual object.

In one or more embodiments, the computer-executable components further comprise an image generation component that generates different images based on the different changes and defined configuration parameters of the X-ray phase contrast imaging system using one or more image generation models (e.g., image generation models as applied to CT image reconstruction). The computer-executable components further comprise an optimization component that determines a preferred improvement to one or more characteristics of the different images and optimizes one or more of the defined configuration parameters to achieve the preferred improvement. Additionally, or alternatively, the optimization component can determine a preferred improvement to one or more characteristics of the different images and optimizes one or more parameters of the one or more image generation models to achieve the preferred improvement.

In some embodiments, elements described in the disclosed systems and methods can be embodied in different forms such as a computer-implemented method, a computer program product, or another form.

### DESCRIPTION OF THE DRAWINGS

FIG. 1 presents an example system that facilitates simulating grating-based X-ray phase contrast imaging systems in accordance with one or more embodiments of the disclosed subject matter.

FIG. 2 presents a three-dimensional (3D) diagram of an example grating-based X-ray phase contrast imaging system in accordance with one or more embodiments of the disclosed subject matter.

FIG. 3 presents a two-dimensional (2D) diagram of an example grating-based X-ray phase contrast imaging system in accordance with one or more embodiments of the disclosed subject matter.

FIG. 4A-4D illustrate different grating-based X-ray phase contrast imaging system configurations in accordance with one or more embodiments of the disclosed subject matter.

FIG. 5 presents a diagram illustrating the relationship between different components of the imaging system and the transverse coherence requirement.

FIG. 6 presents a high-level flow diagram of an example process for simulating performance of a grating-based X-ray phase contrast imaging system in accordance with one or more embodiments of the disclosed subject matter.

FIG. 7 illustrates decomposition of an example virtual object into an absorption object, a phase object, and a small angle scattering object in accordance with one or more embodiments of the disclosed subject matter.

FIG. 8 illustrates a baseline interference pattern plotted as a function of location and amplitude.

FIG. 9 illustrates separately calculating interference patterns attributed to different sub-objects using a projection-based approach in accordance with one or more embodiments of the disclosed subject matter.

FIG. 10 illustrates the phase object projection simulation processing techniques in accordance with one or more embodiments of the disclosed subject matter.

FIG. 11 illustrates calculating the deflection angle based on the refractive index gradient for the phase object.

FIGS. 12A and 12B illustrate influence of aligned and tilted gratings on the X-ray beams of a grating-based X-ray phase contrast imaging system.

FIG. 13 illustrates phase offsets for gratings tilted in their plane.

FIG. 14 illustrates phases offsets for gratings with unmatched periods.

FIG. 15 presents a flow diagram of an example computer-implemented method for simulating performance of a gradient-based X-ray phase contrast imaging system in accordance with one or more embodiments of the disclosed subject matter.

FIG. 16 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosed subject matter is directed to systems, computer-implemented methods, apparatus and/or computer program products that provide computer processing techniques for efficiently and accurately simulating grating-based X-ray phase contrast imaging systems, including CT systems.

Computed Tomography (CT), a method which combines a series of X-ray images taken from different angles and uses computer processing to create cross-sectional images, is the backbone of current diagnostic medical imaging. At the same time, CT has several limitations, such as low soft-tissue contrast, as is necessary for the detection of, for example, oncological lesions, and a limited sensitivity to structural changes in the tissue, which is often necessary for the diagnosis of many different medical conditions.

Grating-based X-ray interferometry offers vast potential for imaging materials and tissues that are not easily visualized using conventional X-ray imaging. Tomographic reconstruction based on X-ray interferometric data provides not only access to the attenuation coefficient of an object, but also the refractive index and information about small angle scattering. However, this improved functionality comes at the cost of longer measurement times, and increased processing time and computational resources, because existing projection-based signal extraction algorithms require not only a single measurement per projection angle but several with precise grating movements in between. This obstacle hinders the adaptation of grating-based interferometry into a continuously rotating gantry of a CT system.

The disclosed subject matter is directed to a computer simulation tool that facilitates testing and simulating new grating-based X-ray phase contrast system design configurations and image generation algorithms that overcome the aforementioned obstacle. In this regard, grating-based X-ray phase contrast imaging techniques are typically based on wave propagation. The complexity of the calculations makes the imaging process very slow, even more so for the many different acquisition angles involved in CT. Simplifications have been proposed using a particle-based approach and ray tracing. This disclosure takes that approach a step further, by no longer following the actual path of the X-ray particle, but virtually accumulating particle deflections from its initial path and weighting them appropriately, without changing the actual path of the particle. This approach is valid in the special case where the particle deflection amount is small with respect to the pixel size (e.g., the deflection amount is less than the detector cell/pixel size), which is generally true for grating-based X-ray phase contrast imaging and various other non-medical X-ray imaging applications.

In addition, the disclosed simulation techniques optimize simulation time by decomposing the scan object (e.g., represented as a virtual object or virtual phantom object) into sub-objects respectively having the different physical properties respectively captured in the three different types of images realized in X-ray phase contrast imaging, that is the absorption image, the phase image and the dark-field image. In this regard, the disclosed techniques decompose the scan object into an absorption object, a phase object and a small angle scattering object, and separately simulate the different interference patterns received at the detector for the different sub-objects. The simulation tool further generates a final interference pattern for the scanned object based on aggregated differences between the interference patterns and a baseline interference pattern received at the detector without the scan object. Respective phase-contrast images (e.g., an absorption image, a phase-contrast image and a dark-field image) can then be reconstructed from the final interference signals simulated for the object at the detector using corresponding image generation algorithms.

With the disclosed techniques the amount of processing time and computational resources involved in the X-ray phase contrast projection simulator are significantly reduced, enabling efficient simulation of X-ray phase contrast projections in CT imaging procedures and development and testing of new corresponding image generation algorithms. The disclosed simulation tool thus enables efficiently and accurately conducting grating-based X-ray phase-contrast imaging trials with different system design configurations and for different types of scan objects in a simulated environment so that they may be eventually implemented in clinical practice in a cost-effective manner.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

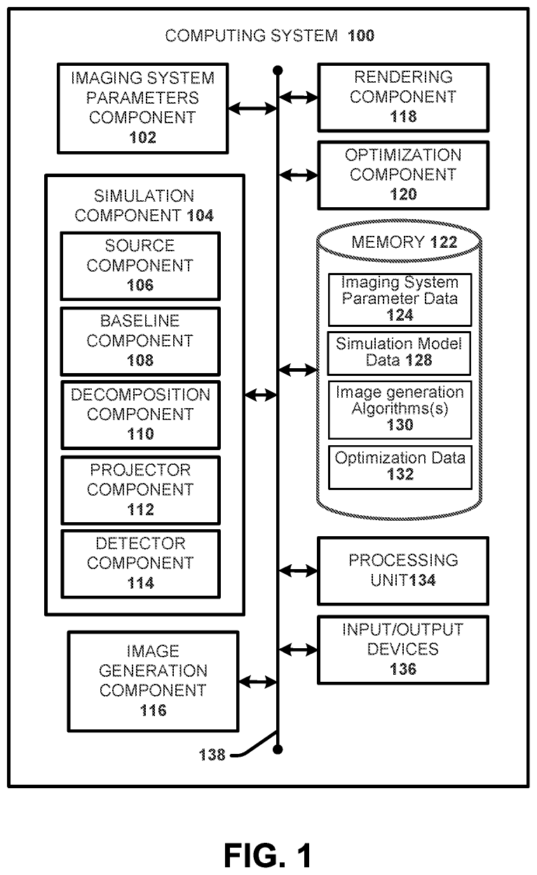

FIG. 1 presents an example computing system (hereinafter 100) that facilitates simulating grating-based X-ray phase contrast imaging systems in accordance with one or more embodiments of the disclosed subject matter. Embodiments of systems described herein can include one or more machine-executable or computer-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

In this regard, system 100 includes various machine-executable components including imaging system parameters component 102, simulation component 104, image generation component 116, rendering component 118, and optimization component 120. These computer/machine executable components (and others described herein) can be stored in memory associated with the one or more machines. The memory can further be operatively coupled to at least one processor, such that the components can be executed by the at least one processor to perform the operations described. For example, in some embodiments, these computer/machine executable components can be stored in memory 122 of system 100 which can be coupled to processing unit 134 for execution thereof. The memory can also store a variety of information that can be used by and/or generated by system 100 to facilitate simulating performance of grating-based X-ray phase contrast imaging systems, including imaging system parameter data 124, simulation model data 128, image generation algorithms 130 and optimization data 132. Examples of said memory 122 and processing unit 130 as well as other suitable computer or computing-based elements, can be found with reference to FIG. 19, and can be used in connection with implementing one or more of the systems or components shown and described in connection with FIG. 1 or other figures disclosed herein.

System 100 further includes input/output devices 136 and a system bus 138 that communicatively and operatively couples the machine executable components (e.g., the imaging system parameters component 102, the simulation component 104, the image generation component 116, the rendering component 118, and the optimization component 120), the memory 122, the processing unit 134 and the input/output devices 136 to one another. The input/output devices 136 can include any suitable input device that facilities receiving user input (e.g., a keyboard, a touchscreen, a touchpad, etc.) and any suitable output device that facilitate rendering data (e.g., a display, a speaker, etc.). In various embodiments, the input/output devices 136 may comprise at least one graphical display capable of rendering image data (e.g., via rendering component) that has been reconstructed by the image generation component 116 based on simulated detector signal data received at a detector of a simulated grating-based X-ray phase contrast imaging system. In this regard, system 100 may be or include any suitable computing device (e.g., a laptop computer, a desktop computer, a virtual computer, a smartphone, a tablet, a server, a quantum computing device, etc.).

The simulation component 104 simulates performance of an X-ray phase contrast imaging system using a simulation model (e.g., represented by simulation model data 128 and/or source component 106, baseline component 108, decomposition component 110, projector component 112 and detector component 114) that models and simulates the physics of the X-ray phase contrast imaging system. In some embodiments, the X-ray phase contrast imaging system may correspond to a conventional X-ray imaging system with phase contrast functionality adapted to scan an object (e.g., an anatomical region of interest (ROI) of the body for medical imaging) from a single scan position and generate a single reconstruction image of the scanned object from a single set of projection data collected at the detector from the single scan position. In other embodiments, the X-ray phase contrast imaging system may correspond to a CT system with phase contrast functionality that captures a plurality of sets of projection data at different scan positions in association with rotation of the gantry and detector (and other imaging system components) around the scanned object (e.g., in association with a 360-degree rotation or a portion thereof, and/or a helical rotation). Still in other embodiments, the X-ray phase contrast imaging system may correspond to a non-medical imaging system. For example, the X-ray phase contrast imaging system may correspond to an imaging system that performs 2D and/or 3D image signal acquisition and image generation for non-medical applications (e.g., non-destructive testing) wherein the object may be rotated (instead of the gantry being rotated).

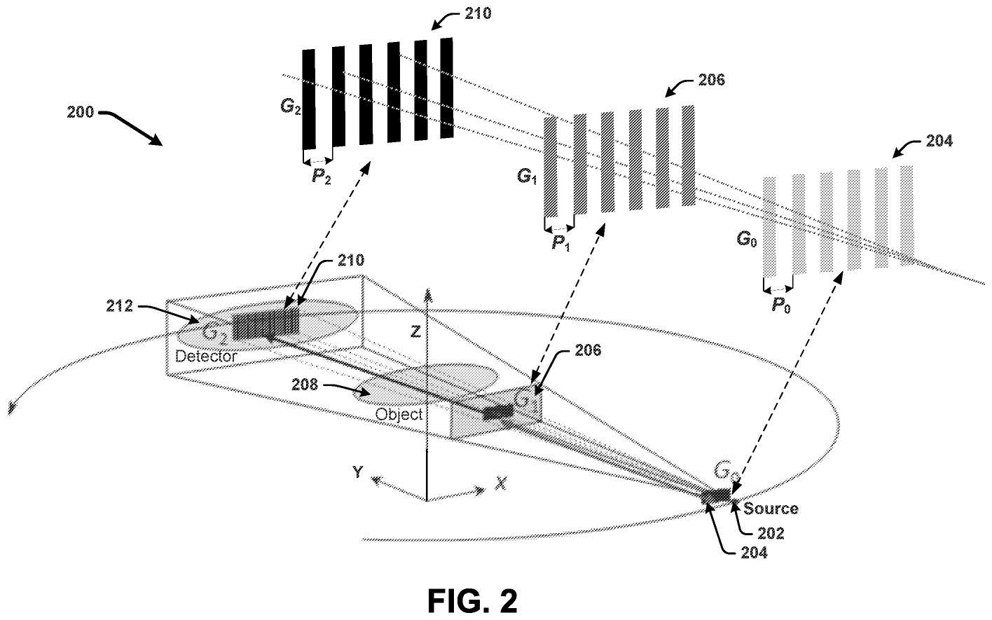

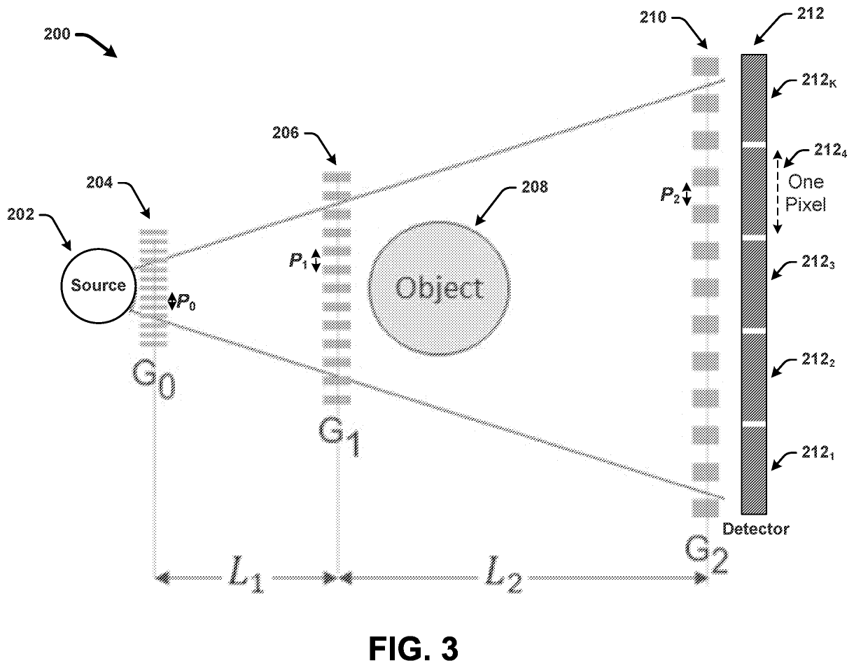

FIG. 2 presents a 3D diagram of an example grating-based X-ray phase contrast imaging system 200 (hereinafter imaging system 200) in accordance with one or more embodiments of the disclosed subject matter. FIG. 3 presents a 2D diagram of imaging system 200.

With reference to FIGS. 2 and 3, in accordance with conventional X-ray and CT imaging systems, the X-ray phase contrast imaging system 200 includes an X-ray source 202 and an X-ray detector 212 positioned opposite the X-ray source 202. The X-ray source 202 emits a beam of X-rays towards the X-ray detector 212 which is composed of a plurality of detector elements 2121-K (e.g., typically arranged in 2D array). Each of the detector elements 2121-K corresponds to one pixel of the projected images. The object 208 to be scanned (e.g., an anatomical region of interest (ROI) of the body for medical imaging) is positioned between the X-ray source 202 and the X-ray detector 212 and radiated with an X-ray beam emitted by the X-ray source 202. The X-ray source 202 projects X-ray photons through the object 208 (e.g., along with the gratings as further discussed below) and the X-ray detector 212 on the opposite side of the object 208 measures X-ray attenuations of the photons by the object (e.g., different tissues inside the body) based on the amount (e.g., based on both the number and the energy) of photons arriving at the detector elements, referred to as projection signals or simply projections. The physical density of the respective physical components of the object (e.g., tissues, bones, and other anatomical features as applied to medical imaging objects) is proportional to the absorption/attenuation of the X-ray beam.

A conventional X-ray absorption image can be reconstructed based on the amount of X-ray photons received at the detector elements after passing through the object 208 being scanned. In this regard, both the amount of photons and the energy of the photons are measured in association with generating X-ray absorption images from the detector signals. In a standard (energy-integrating) detector, the signal measured adds the energy of all photons detected during a detection interval. In energy-sensitive detection schemes (e.g., photon-counting detectors), photons at different energies can be weighted differently. In embodiments in which the imaging system 200 corresponds to a CT system, the X-ray source 202 and the X-ray detector 212 (e.g., along with the gratings as further discussed below) are rotated around the object 208 about a rotation axis (e.g., the Z-axis as depicted in FIG. 2) to capture a plurality of projection images at different capture positions or projection angles. The multiple X-ray projections taken from different angles are then processed using CT reconstruction algorithms (e.g., forward projection or Radon transform, back-projection or inverse Radon transform, filtered back-projection, etc.) to convert the projection images into grayscale images to produce tomographic (cross-sectional) images (reconstructed “slices”) of the object scanned.

In both conventional X-ray and CT imaging systems, the obtained contrast of the reconstructed absorption images relies on differences in the attenuation cross-section of the constituents of the object 208. Conventional X-ray and CT imaging absorption images yield excellent results for structures that are highly absorbing with major differences in absorption, such as osseous structures and the lungs. The high contrast is obtained due to the fact that usually bony structures are embedded in weakly absorbing tissue, such as muscles or adipose tissue of the human body. In contrast, for structures with relatively similar absorbing characteristics such as different types of soft tissue, conventional X-ray-based techniques achieve relatively low contrast resolution. However, these tissues are of particular interest for medical diagnostics as numerous pathologic processes affect soft tissues (e.g., in the setting of cancer or vessel structures and others).

Phase shift functionality rooted in a grating-based approach has been used to enhance X-ray based imaging technology to obtain additional images (e.g., in addition to the absorption image, also referred to as the attenuation image, the offset image and similar terms) that provide greater contrast for tissues with similar absorption characteristics. The additional images that can be obtained include a phase-contrast image (also referred to as a differential phase-contrast image, a phase shift image, and similar terms), and a dark-field image (also referred to as a scattering image, small angle scattering image, and similar terms). These additional images are based on how the X-rays are deflected by the object 208 along their wave path.

In this regard, X-ray phase-contrast imaging or phase-sensitive X-ray imaging is a general term for different technical methods that use information concerning changes in the phase of an X-ray beam that passes through an object in order to create its images. X-ray phase-contrast imaging offers the potential for increased soft-tissue contrast due to a more significant difference between different types of soft tissue in the refractive index than in the attenuation coefficient. Standard X-ray imaging techniques like radiography and CT rely on a decrease of the X-ray beam's intensity (attenuation) when traversing the sample, which can be measured directly based on the intensity of the projection signals received at the detector 112. However, in phase contrast X-ray imaging, the X-ray beam's phase shift caused by the object is not measured directly, but is transformed into variations in intensity, which then can be recorded by the detector 112. In particular, when electromagnetic waves enter a medium with a different refractive index, they are bent, a phenomenon that is well known for visible light. The same applies to X-rays, with the small difference that they are usually bent in the opposite direction of visible light, as the X-ray phase velocity of most objects is faster than the speed of light. The angle of diffraction depends on the gradient of the refractive index within each voxel of the object, and not on the absolute value of the refractive index. This implies that the phase shift measurement has high sensitivity to small features, but rather low sensitivity to large features. The angle of diffraction is generally determined by comparing the interference pattern received at the X-ray detector 212 with the object to the interference pattern without the object (referred to herein as the baseline interference pattern).

X-ray dark field imaging offers a tool which can assess structural changes within a sample as the contrast in X-ray dark field imaging is created by small angle scattering at microstructures of a much smaller scale than the spatial resolution of the imaging system 200. For visible light, dark field imaging usually refers to the imaging of strongly scattered light, with the illuminating light being filtered out from the final image. For X-rays, dark field imaging usually refers to small angle scattering, where a microstructure within a single pixel leads to a multitude of phase-shifted interference patterns within that pixel. The addition of those interference patterns with multiple phase shifts then leads to a visibility reduction of the overall interference pattern received at the detector 112. For homogeneous objects that show no or negligible small-angle scattering, the dark field signal is close to zero, whereas strongly scattering samples yield a significant signal. This way, the dark-field images reveal sub-resolution structural information on the nanometer and micrometer length scale that is inaccessible using conventional attenuation X-ray imaging.

A variety of X-ray phase-contrast and X-ray dark field imaging techniques have been developed, all of which are based on the observation of interference patterns between diffracted and undiffracted waves. The most common techniques are crystal interferometry, propagation-based imaging, analyzer-based imaging, edge-illumination and grating-based imaging. The disclosed subject matter is directed to the grating-based approach. The grating-based approach is based on the introduction of a grating interferometer into the beam path so that the signal measured on the X-ray detector 212 is sensitive to X-ray attenuation, refraction and ultra-small-angle scattering. The fundamental idea of the grating-based X-ray imaging approach is to evaluate the local changes of the oscillation in the baseline interference pattern received at the X-ray detector 212 induced by an object 208 and determine several imaging signals based on the changes, namely the conventional transmission image, the differential phase-contrast (or refraction) image, and the dark-field (or scattering) image.

In this regard, imaging system 200 includes a grating interferometer positioned between the X-ray source 202 and the X-ray detector 212 that employs a three-grating topology comprising a source grating 204 (G₀), a second grating 206 (G₁) and a third grating (210) G₂. Each of the three gratings comprise an array of e.g., vertically oriented (e.g., parallel to the Z-axis) and periodically repeating slits or apertures formed within an absorbing material (making them an absorption grating) or phase-shifting material (e.g., making them a phase grating). For example, the respective gratings are typically made from silicon (Si) wafers using standard photolithography techniques, and subsequent electroplating with to fill the grooves with gold for G₀ and G₂). The periodicity of the respective gratings refers to the size of the slits or apertures of the respective gratings, respectively noted as P₀ for G₀, P₁ for G₁, and P₂ for G₂. The periodicity of the respective gratings can vary however the periodicity P₂ is typically significantly smaller than the pixel size of the detector pixels. For example, in various embodiments, the periodicity of G₂ (i.e., the final grating directly in front of the detector, and therefore that which has to be compared to the detector cell/pixel size) is about 10-30 micrometers (μm) while the pixel/cell size is about 1 millimeter (mm). With this configuration, the size of the X-rays that pass through G₂ is smaller than the pixel size. In various embodiments, the periodicity of each of the different gratings may be the same or different. For example, in some embodiments, the periodicity of G₂ is larger than that of G₁. (i.e., P₂ is greater than P₁). Other configurations are envisioned.

In accordance with the embodiment illustrated in FIG. 2, the source grating (G₀) is placed close to the X-ray source 202 and serves as an aperture mask with transmitting slits. The source grating G₀ creates an array of periodically repeating line sources and effectively allows the use of relatively large X-ray sources (e.g., square-millimeter-sized) without compromising the coherence requirements of the arrangement formed by G₁ and G₂, as discussed in greater detail below. In some embodiments, the source grating G₀ can be omitted and replaced with one or more independent point or line sources. The image contrast itself is formed via the combined effect of the two gratings G₁ and G₂. The second grating 206 (G₁) acts as a mask and imprints periodic phase modulations onto the incoming wave field. Through the Talbot effect, the phase modulation is transformed into an intensity modulation in the plane of the second grating 210 (G₂) forming a linear periodic fringe pattern, referred to as the Talbot carpet or Talbot effect, perpendicular to the optical axis (i.e., the Y-axis) and parallel to the rotation axis (i.e., the Z-axis). The third grating G₂ is positioned close to the X-ray detector 212 and the object 208 is positioned between the second grating G₁ and the third grating G₂. This is good design for CT imaging. In other embodiments, (e.g., for other applications aside from CT), the object 208 may be positioned between G₀ and G₁. The periodicity of the second grating G₁ and the third grating G₂ may be the same, substantially similar or different. The distance between the source grating G₀ and G₁ is indicated as L₁ and the distance between G₁ and the G₂ is indicated by L₂. The second grating G₁ and the third grating G₂ can be positioned relative to one another so that the apertures of the respective gratings are perfectly aligned or slightly offset from one another to increase the contrast of the deflected particles that pass through or do not pass through the third grating G₂.

As noted above, in various embodiments, the baseline interference pattern (i.e., the pattern observed at the detector of imaging system 200 without the object 208) received at the X-ray detector 212 corresponds to a Talbot carpet. In this regard, a grating (i.e., G₁) illuminated by monochromatic light produces a Talbot carpet, in which the initial pattern is reimaged after the Talbot distance Z_{T}. The Talbot distance Z_{T} depends on both the distance P between the slits and the wavelength λ of the light. The Talbot distance Z_{T} serves as a constraint on the design of the imaging system 200 because we need to ensure that L₁ and L₂ are close to a Talbot distance or a multiple thereof, to ensure the reimagined pattern at the detector corresponds to the initial pattern created by G₀ and G₁ respectively. In this regard, the Talbot distance Z_{T} for G₁ can be calculated from the distance P₁ between the slits of G₁, and the wavelength A of the light in accordance with Equation 1 below for an absorption grating and Equation 2 below for a phase shift grating. The Talbot distance Z_{T} for G₁ can similarly be calculated from the distance P₁ between the slits of G₁, and the wavelength λ of the light in accordance with Equation 1 below for an absorption grating or π/2-shifting phase grating, and Equation 2 below for a π-shifting phase shift grating.

$${z}_{T} = \frac{{p}_{1}^{2}}{2 λ} . \qquad Equation 1$$

$${z}_{T} = \frac{{p}_{1}^{2}}{8 λ} . \qquad Equation 2$$

In accordance with cone beam geometry (e.g., that employed by system 200 and the like), the Talbot distances applied for L₁ and L₂ need to be scaled with the magnification factor. In this regard, the distance P₂ between the gratings of G₂ can be calculated in accordance with Equation 3.

$${p}_{2} = {p}_{1} \cdot \frac{{L}_{1} + {L}_{2}}{{L}_{1}} . \qquad Equation 3$$

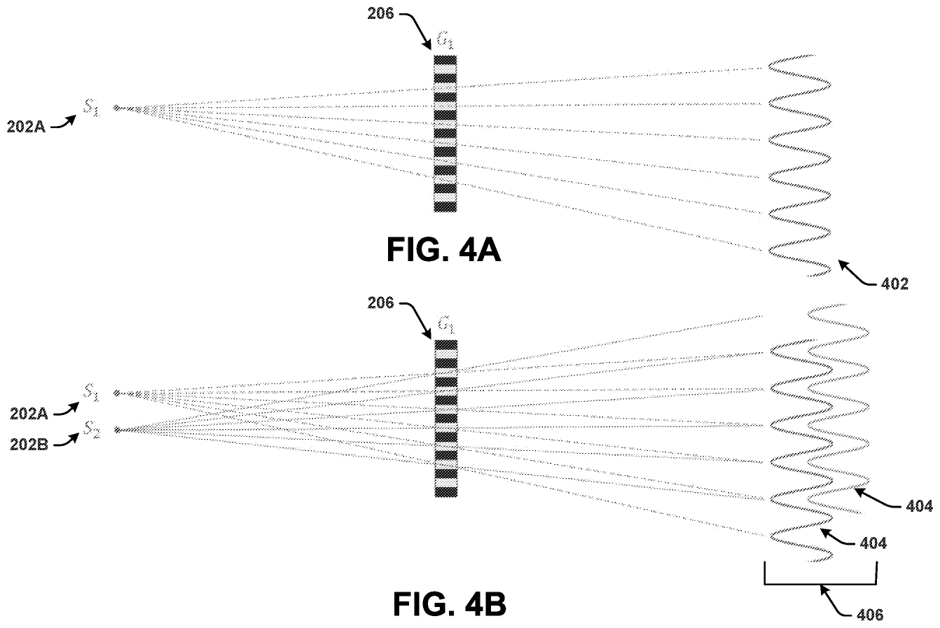

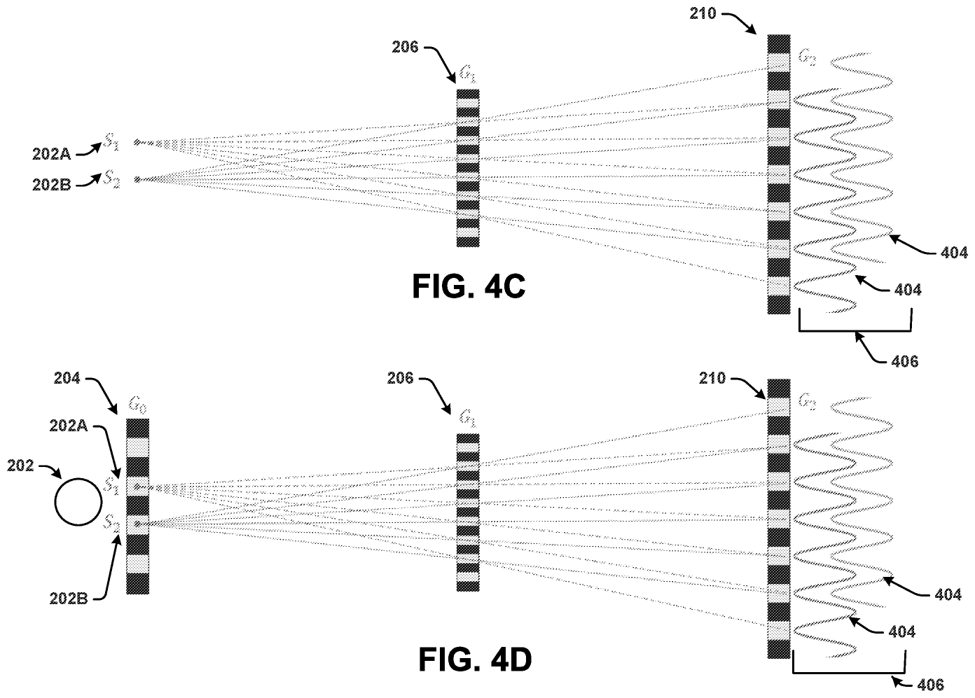

As noted above, in some embodiments, the X-ray source 202 may comprise one or more point or line sources, in which case the source grating 204 (G₀) may be omitted. For a larger source, the source grating 204 (G₀) can be included in the imaging system 200 to create multiple (e.g., two or more) incoherent line sources and the interference fringes created by each of the line sources overlap on the detector, as illustrated in FIGS. 4A-4D.

FIGS. 4A-4D illustrate different grating-based X-ray phase contrast imaging system configurations in accordance with one or more embodiments of the disclosed subject matter. The sources illustrated in FIGS. 4A-4D (i.e., S₁ and S₂) can be interpreted as being either points, or lines that are orthogonal to the page. FIG. 4A illustrates a first configuration that employs a single point source (first point source 202A (S₁)) and grating G₁. As illustrated in FIG. 4A, the first point source 202A emits an X-ray beam through grating G₁, which results in the generation of a first interference pattern 402 to be received at the detector. FIG. 4B illustrates a second configuration that adds a second point source 202B (S₂) to create more flux. The second point source 202B creates a second interference pattern 404, independent of the first interference pattern 402, that superimposes with the first interference pattern 402 to interference pattern 406. FIG. 4C illustrates addition of a second grating G₂ after the first grating G₁ to the configuration illustrated in FIG. 4B. The second grating G₂ is positioned in front of the detector (not shown) and allows for the interference pattern 406 to be detected. In various embodiments, the two point sources 202A (S₁) and 202B (S₁) can be effectively created from a single X-ray source 202 using a grating G₀ as illustrated in FIG. 4D. In this regard, the source grating G₀ creates an array of individually coherent, but mutually incoherent X-ray beam sources. A phase object in the beam path causes a slight refraction for each coherent subset of X-rays, which is proportional to the local differential phase gradient of the object. This small angular deviation results in changes of the locally transmitted intensity through the combination of gratings G₁ and G₂.

In accordance with embodiments in which multiple (e.g., two or more) point sources are used (or created via G₀), the phenomenon of transvers coherence must be accounted for in association with designing the imaging system 200 to achieve quality dark field images. Transverse coherence is the lateral distance along a wavefront over which there is a complete dephasing between two waves, of the same wavelength, which originate from two separate points in space.

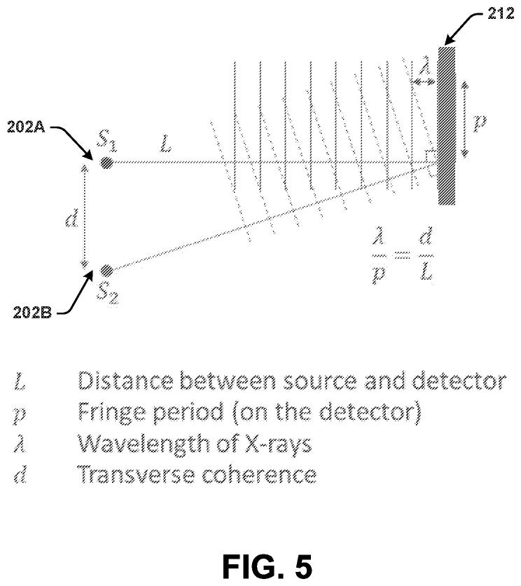

FIG. 5 presents a diagram illustrating the relationship between different components of the imaging system 200 and the transverse coherence requirement. The transverse coherence requirement is based on the distance L between the source and the X-ray detector 212, the fringe period p on the detector, the wavelength λ of the X-rays, and the transverse coherence distance d of the X-ray source (also referred to as the autocorrelation distance of the X-rays and the focal spot size). The transverse coherence requirement restricts the relationship between these parameters in accordance with Equation 4.

$$d = \frac{λ L}{p} . \qquad Equation 4$$

In accordance with the transverse coherence requirement and Equation 4, if the source has transverse coherence length of d or larger, the interference fringes at the X-ray detector 212 will wash out. Dark field is highly sensitive only to features that are in the same order of magnitude as the transverse coherence distance d of the X-rays. The sensitivity of the dark field contrast depends on the ratio between the size of the microstructure, and the transverse coherence length d.

Phase contrast dark field only requires the amplitude, not the phase. Therefore, dark field is much more robust to misalignment, and even to vibrations, as long as the vibration frequency is low compared to the view acquisition frequency.

With reference again to FIG. 1 in view of FIGS. 2-5, in accordance with various embodiments, the simulation component 104 models and simulates the performance of a grating-based X-ray phase contrast imaging system, such as imaging system 200 and similar systems, and determines the baseline interference signal received at the X-ray detector 212 of the imaging system without a scanning object. The simulated performance of the grating-based X-ray phase contrast imaging system can be based on one or more predefined imaging system parameters (e.g., included in imaging system parameter data 124), one or more user defined imaging system parameters (e.g., received via manual input via imaging systems parameters component 102 and input/output devices), and/or one or more simulation component 104 determined parameters as constrained based on Equations 1-4. In this regard, the imaging system parameters can include, but are not limited to, the distance L between the source and the X-ray detector 212, the fringe period p on the detector, the wavelength A of the X-rays, the energy of the X-rays, the spectral filter parameters (e.g., material and thickness), the transverse coherence distance d of the X-ray source, the distances between the gratings (e.g., L₁ and L₂), the grating periods (e.g., P₀, P₁ and P₂), the grating profiles (e.g., triangular, rectangular, with different duty cycles), the alignment between the gratings (e.g., between G₁ and G₂), and the configuration of the detector (e.g., the detector cell/pixel size, and the number and arrangement of the detector elements in the detector array). It should be appreciated that in X-ray imaging systems, the X-ray tubes do not just create a single wavelength, but an entire spectrum of different wavelengths (i.e., many different wavelengths). The width of this spectrum can be reduced through spectral filtering, but this also reduces X-ray flux. Parameters that control the spectrum are the electron energy (energy of the electrons impinging onto the target and creating X-rays), as well as the material (e.g., typically, aluminum (Al), copper (Cu) or tin (Sn)) and the thickness of the spectral filter. As noted above, the sensitivity of the dark field contrast depends on the ratio between the size of the microstructure of the object to be scanned and the transverse coherence length d. Thus, in various embodiments, the simulation component 104 can set and adjust one or more parameters of the modeled imaging system based on the size of the microstructure of the object to be scanned. The simulation component 104 can further model the imaging system under a variety of different configurations and for a variety of different scan objects with different properties as constrained based on Equations 1-4.

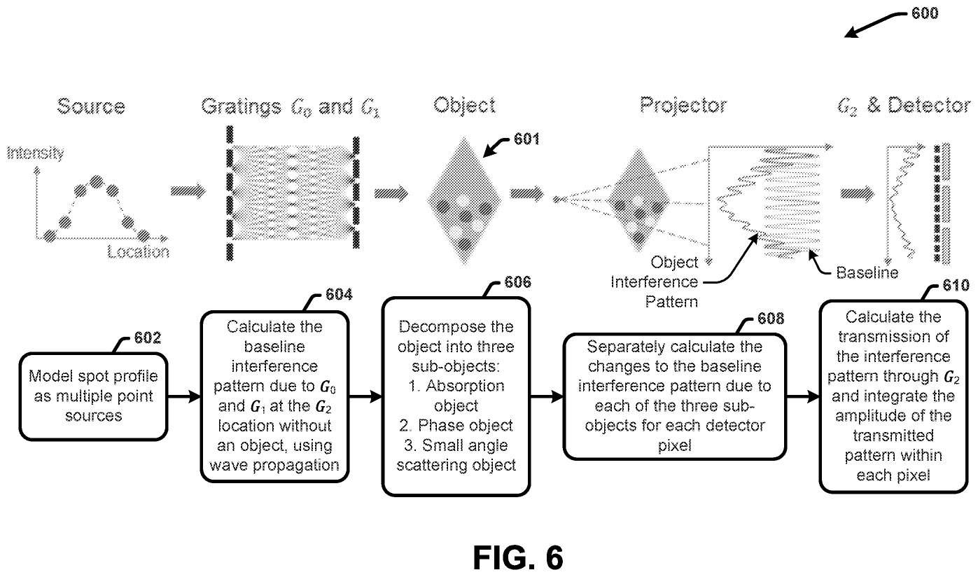

The simulation component 104 further models the performance of the imaging system under the defined imaging system configurations in association with simulated scanning of a virtual object (i.e., a phantom) corresponding to a real object with different physical properties. The simulated performance of the imaging system with the object may correspond to performance of an X-ray scan and/or a CT scan. In this regard, the simulation component 104 determines the resulting interference pattern received at the X-ray detector 212 based on simulated projection of the X-ray beam through the virtual object, wherein the resulting interference pattern is determined based on estimated changes to the baseline interference pattern attributed to the scanned object. To facilitate the simulations, the simulation component 104 can include source component 106, baseline component 108, decomposition component 110, projector component 112 and detector component 114. Additional details regarding the specific features and functionalities of these components are discussed below with reference to FIG. 6.

The image generation component 116 can then generate different images of the scanned object based on the resulting interference pattern received at the detector, including an absorption image (also referred to as an attenuation image), a phase contrast image (also referred to as a phase shift image and phase gradient image), and a dark field image (also referred to as a small angle scattering image) based on the defined configuration parameters of the X-ray phase contrast imaging system and using one or more image generation algorithms or models (e.g., image generation algorithms 130). In some embodiments, the rendering component 118 can render the different images on a graphical display (e.g., included in the input/output device 136) to a system operator for manual review.

Additionally, or alternatively, the optimization component 120 can determine a preferred improvement to one or more characteristics of the different images based on analysis of the different images using defined optimization criteria (e.g., included in the optimization data 132) and optimizes one or more of the defined configuration parameters of the X-ray phase contrast imaging system design (e.g., determines one or more adjustments to the imaging system design configuration) to achieve the preferred improvement. For example, the disclosed simulation techniques can be employed to test out different imaging system design configurations (e.g., with respect to grating period, position and alignment, focal spot size, X-ray wavelength, etc.) for different scan objects and evaluate the reconstructed images generated based on the detector signals received under the different design configurations. The optimization component 120 can further evaluate the reconstructed images (e.g., the absorption images, the phase-contrast images, and the dark-field images) using defined image optimization criteria to determine how to adjust one or more parameters of the system design configuration to improve the quality of the reconstructed images. In some embodiments, the simulation component 104, the image generation component 116 and the optimization component 120 can perform an iterative process of simulating the scanning and reconstruction process and adjusting one or more of the system design configurations based on analysis of the reconstructed images until the quality of the reconstructed images becomes satisfactory (e.g., relative to defined acceptable criteria for the respective images). To facilitate this end, the optimization component 120 can employ one or more supervised, semi-supervised and/or unsupervised machine learning techniques.

Still in other embodiments, the optimization component 120 can determine a preferred improvement to one or more characteristics of the different images based on analysis of the different images using defined optimization criteria (e.g., included in the optimization data 132) and optimizes one or more parameters of the one or more image generation algorithms 130 to achieve the preferred improvement. For example, the disclosed simulation techniques can be employed to test out different image generation algorithms for generating reconstructed images (e.g., absorption images, phase-contrast images and dark-field images) based on the simulated interference signals received at the detector in association with performance of an X-ray and/or CT scan (with a rotating gantry). In this regard, the optimization component 120 can evaluate the reconstructed images (e.g., the absorption images, the phase-contrast images, and the dark-field images) using defined image optimization criteria to determine how to adjust one or more parameters of the image generation algorithms under the same system design configuration to improve the quality of the reconstructed images. In various embodiments, the optimization component 120 can perform an iterative optimization process wherein one or more imaging system configuration parameters and reconstruction algorithm parameters are iteratively adjusted until the final image quality achieved based on a combination of the system configuration parameters and the image generation algorithms is satisfactory.

FIG. 6 presents a high-level flow diagram of an example process 600 for simulating performance of a grating-based X-ray phase contrast imaging system in accordance with one or more embodiments of the disclosed subject matter. With reference to FIGS. 1-6, in various embodiments, the grating-based phase contrast imaging system corresponds to imaging system 200. Process 600 involves modeling the physics of the grating-based X-ray phase contrast imaging system under defined imaging system parameters without a scan object to determine the baseline interference pattern (e.g., via the baseline component 108). Thereafter, the simulation component 104 simulates performance of an imaging scan by the modeled imaging system under the defined imaging system parameters with a scan object (a virtual scan object modeled by the decomposition component 110 as discussed below) and determines the resulting interference pattern received at the detector based on changes to the baseline interference pattern attributed to the scan object. The image generation component 116 can then generate reconstructed images of the scan object based on the resulting interference pattern and using one or more image generation algorithms 130, wherein the reconstructed images can include an absorption image (e.g., a standard absorption image), a phase-contrast image, and a dark field image.

In some embodiments, the performance of the imaging scan corresponds to performance of a conventional X-ray from a single scan angle/position. In other embodiments, the performance of the imaging scan corresponds to performance of a CT scan, wherein the resulting interference pattern is determined for the object for each scan angle/position in association with rotation of imaging system (e.g., the X-ray source 202, the detector 112 and the grating interferometer around the scan object). In both of these embodiments, the simulation component 104 determines the baseline interference pattern once prior to simulating the imaging scan with the scan object based on the applied imaging system parameters (e.g., the distance L between the X-ray source 202 and the X-ray detector 212, the fringe period p on the detector, the wavelength A of the X-rays, the transverse coherence distance d of the X-ray source, the distances between the gratings (e.g., L₁ and L₂), the grating periods (e.g., P₀, P₁ and P₂), the alignment between the gratings (e.g., between G₁ and G₂), and the configuration of the detector (e.g., the detector cell/pixel size, and the number and arrangement of the detector elements in the detector array). In this regard, the baseline component 108 does not calculate a separate baseline interference pattern for each scan angle/position of a CT scan, thereby minimizing the amount of computational resources and processing time attributed to the entire simulation process. However, the simulation component 104 can test different imaging system configurations tailored for different types of objects with different physical properties corresponding to different anatomical ROIs and anatomical structures. In these scenarios, the baseline component 108 determines a new baseline interference pattern for each of the different imaging system configurations (i.e., with different imaging system parameter values).

In this regard, in accordance with process 600, at 602 the source component 106 models the spot profile of the grating-based X-ray phase contrast imaging system X-ray source (e.g., imaging system 200 or the like) as multiple point sources (e.g., two or more), as demonstrated in FIGS. 4B-4D in accordance with Equations 1-4 under one or more defined imaging system parameters (e.g., values of: the distance L between the source and the X-ray detector 212, the fringe period p on the detector, the wavelength λ of the X-rays, the energy of the X-rays, the spectral filter parameters (e.g., material and thickness), the transverse coherence distance d of the X-ray source, the distances between the gratings (e.g., L₁ and L₂), the grating periods (e.g., P₀, P₁ and P₂), the grating profiles (e.g., triangular, rectangular, with different duty cycles), the alignment between the gratings (e.g., between G₁ and G₂), and the configuration of the detector (e.g., the detector cell/pixel size, and the number and arrangement of the detector elements in the detector array). At 604, the baseline component 108 calculates the baseline interference pattern due to G₀ and G₁ at the G₂ location under the defined imaging system parameters without an object using wave propagation and in accordance with Equations 1-4 (e.g., which corresponds to a Talbot carpet illustrated above 604).

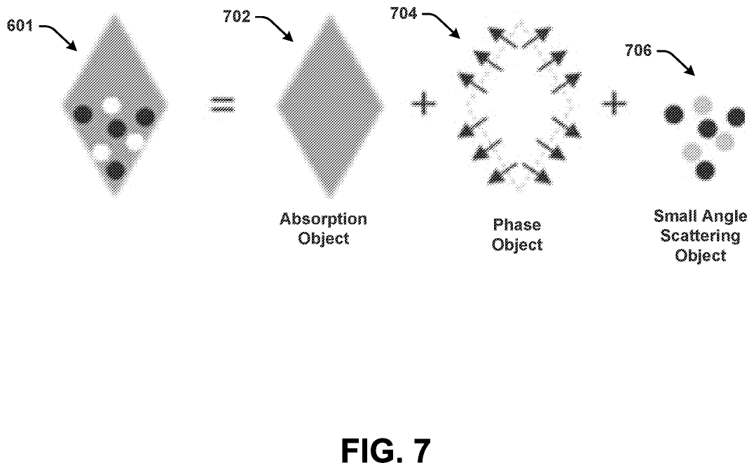

At 606, the decomposition decomposes a voxelized virtual object 601 to be scanned in the simulation into three different sub-objects having different physical properties, wherein the different sub-objects are also voxelized in 3D. The virtual object can correspond to any 3D object with an X-ray absorption profile, refractive properties and particle features. As described in greater detail below, the absorption characteristics, the refractive properties and the particle features of the respective objects are determined/modeled for each voxel of each sub-object. In particular, as illustrated in FIG. 7, the decomposition component models the virtual object as three separate sub-objects respectively corresponding to an absorption object 702 that accounts for the X-ray absorption profile of the object's components, a phase object 704 that accounts for the phase-shift deflection characteristics of the object, and a small angle scattering object 706 that accounts for the small-angle scattering properties of the object attributed to the microstructure of the object's particles.

With reference again to FIG. 6, At 608, the projector component 112 then separately calculates or models different changes to the baseline interference pattern due to each of the three sub-objects for each detector pixel. The projector models the signals received at the detector (i.e., what the detector sees). In particular, the projector component 112 separately simulates projection of the X-ray beam from the modeled point sources through the modeled imaging system (e.g., gratings G₀ and G₁) and the absorption object, the phase object and the small angle scattering object (i.e., performing three separate sub-simulations) and independently determines how each sub-object changes the baseline interference pattern. The different changes to the baseline interference pattern respectively correspond to an absorption change attributed to the absorption object, a phase shift change attributed to the phase object, and a dark field change attributed to the small angle scattering object. The changes are calculated for each pixel of the detector array.

For example, the projector component 112 determines how the absorption object changes the baseline interference pattern independently from the phase object and the small angle scattering object for each detector pixel. The changes to the baseline interference pattern attributed to the absorption object include an amount decrease in total amplitude of the baseline interference signal at each detector pixel (e.g., due to absorption of the X-ray beam photons by respective voxels of the absorption object). The projector component 112 also determines how the phase object changes the baseline interference pattern independently from the absorption object and the small angle scattering object for each detector pixel. The changes to the baseline interference pattern attributed to the phase object include a shift or displacement amount and direction of the baseline interference pattern at each detector pixel attributed to refraction or deflection (i.e., a change in direction of the X-rays attributed to refraction) of the X-ray beam photons by respective voxels of the phase object as function of the refractive index properties of the respective voxels. The projector component 112 also determines how the small angle scattering object changes the baseline interference pattern independently from the absorption object and the phase object for each detector pixel. The changes to the baseline interference pattern attributed to the small angle scattering object include a decrease in visibility and/or modification to the shape of the baseline interference pattern at each detector pixel attributed to small angle scattering of the X-ray beam photons by respective voxels of the phase object as function of the scatter angle standard deviation of the respective voxels.

At 604, the detector component 114 further calculates the transmission of the interference pattern through G₂ and integrates the amplitude of the transmitted pattern within each detector pixel. In this regard, for each detector pixel, the detector component 114 aggregates the changes to the baseline interference pattern attributed to each sub-object (i.e., the absorption object, the phase object and the small angle scattering object), resulting in a final interference pattern for the virtual object. In other words, the detector component 114 combines the different changes separately determined for each of the sub-objects together to generate an aggregated interference pattern for the virtual object. The idea behind this is similar to describing a movement in a 3D coordinate system (e.g., having an x-axis, a y-axis and a z-axis) defined by a movement in the x direction, a movement in the y direction and a movement in the z direction, and then applying the three movements to the coordinates of the point that has to be moved, to calculate its final position. As applied to phase-shift imaging systems, the three movements include a change in the phase, the offset and the amplitude of the baseline interference pattern.

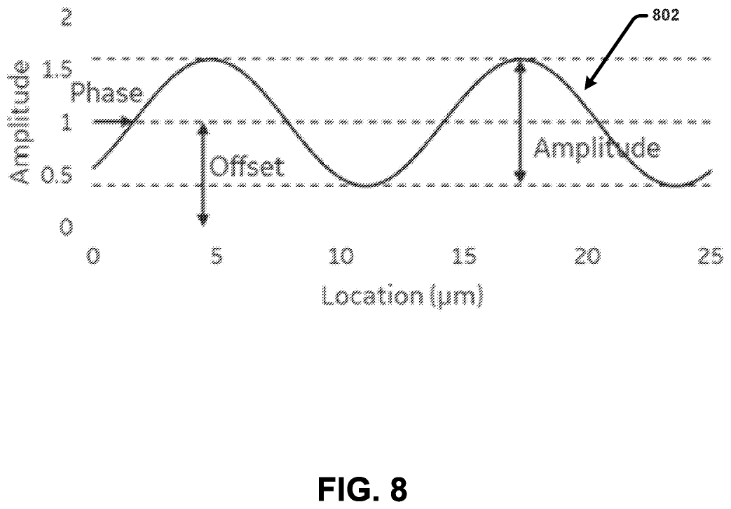

For example, FIG. 8 illustrates a baseline interference pattern 802 plotted as a function of location and amplitude. In this example, the baseline interference pattern 802 is depicted as a sinusoidal curve, which is an approximation. The baseline interference pattern can be defined by three components, a phase value, an offset value and an amplitude value. The changes to the baseline interference pattern by the 3 sub-objects can be applied in a similar way to a non-sinusoidal baseline interference pattern. In this regard, assume relative to a single pixel of the plurality of detector pixels, the baseline interference pattern can be defined by a first phase value (Phase1), a first offset value (Offset1) and a first amplitude value (Amplitude1). Now assume the absorption object changes/scales the first offset value and the first amplitude value (by a first factor); the phase object changes the first phase value (by a second term), and the small angle scattering object changes/scales the first amplitude value by a third factor. The detector component 114 aggregates all the changes to the first phase value, the first offset value and the first amplitude value together for that detector pixel to generate a final interference pattern for that detector pixel which can be described by a second phase value (Phase2) a second offset value (Offset2) and a second amplitude value (Amplitude2). This process is performed for each pixel of the plurality of detector pixels.

It should be appreciated that while the “vertical” parameters (offset and amplitude) are scaled, the phase parameter will not be scaled, but a fixed number (“a phase shift value”) will be added to the initial phase value.

In this regard, for a phase object, each voxel can be represented by a single number (i.e., a scalar value), whereas in a gradient field each voxel is generally represented by a 3-dimensional vector (to specify the vector in 3D space). In some embodiments, the phase object may correspond to a phase gradient object represented by a 3-dimensional vector to specify the vector in 3D space. With these embodiments, the simulation component 112 can measure and calculate the gradient for each voxel.

The disclosed techniques for aggregating the effects of the different sub-objects on the baseline interference pattern, particularly with respect to the phase object and the small angle scattering object, are rooted in the observation that although X-ray phase contrast imaging is based on the fact that X-rays are refracted by the scanned objects (i.e., they change direction), in many practical cases, the angular change of the X-rays is so small that the resolution (i.e., the pixel size) of the X-ray detector 212 is not large enough for directly measuring the refraction distance. A common way to overcome this is to introduce gratings that have a period that is much smaller than the pixel size.

The disclosed techniques employ a grating period and focal spot size configuration that assumes the X-ray deflection is small with respect to the pixel size of the detector, which is generally true for medical X-ray phase contrast imaging. The disclosed techniques further assume that the interference pattern incident onto each pixel is made up of a large number of grating periods, and that the refraction distance of the X-ray beams is in the order of magnitude of this interference pattern period, while being small compared to the pixel size. Therefore, the disclosed techniques can model the X-ray path as being a straight path through the object (e.g., as opposed to being refracted and changing direction), as the majority of the refracted X-rays remain within the same pixel. In this regard, the projector component 110 calculates the changes to the baseline interference pattern attributed to the phase-gradient object and the phase-shift object without following the deflected path of the X-ray particle and without determining its deflected position on the detector. On the contrary, the projector component 110 estimates all (e.g., one or more) particle deflection amounts from its initial path (e.g., corresponding to the direct path of the particle from the source to the detector without any deflection caused by the object) and aggregates the individual deflection amounts to generate an aggregated deflection amount binned/summed for each pixel while weighting the deflection amounts based on their deflection distances (e.g., the distance between the deflection location and the detector).

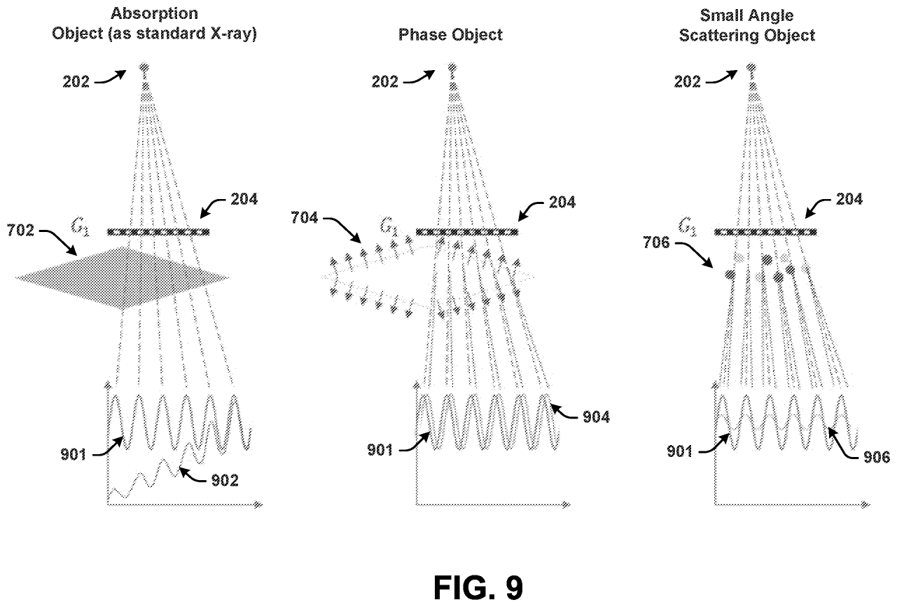

FIG. 9 illustrates separately simulating the interference patterns attributed to different sub-objects using a projection-based approach in accordance with one or more embodiments of the disclosed subject matter. An example baseline interference pattern 901 (i.e., the interference pattern without the object) is illustrated for each sub-object, which is the same for each sub-object. Interference pattern 902 presents an example interference pattern attributed to the absorption object 702 alone, interference pattern 904 presents an example interference patter attributed to the phase object 704 alone, and interference pattern 906 illustrates an example interference pattern attributed to the small angle scattering object 706 alone. Each of the sub-object's interference patterns are superimposed over the baseline interference pattern 901 to illustrate the differences between the respective patterns. As illustrated via comparison of interference patterns 902, 904 and 906, each of the different sub-objects change the baseline interference pattern differently. For example, the absorption object 702 decreases the total amplitude of the baseline interference pattern 901, the phase object 704 displaces or offsets the baseline interference pattern, and the small angle scattering object 706 decreases the visibility of the baseline interference pattern 901 and modifies the shape of the baseline interference pattern 901.

In accordance with the disclosed techniques, for each detector pixel, the projector component 112 determines absorption differences between the baseline interference pattern 901 and interference pattern 902, phase shift differences between the baseline interference pattern 901 and interference pattern 904, and small angle scattering differences between the baseline interference pattern 901 and interference pattern 906. For each detector pixel, the detector component 114 then aggregates the respective differences to the baseline interference pattern together (e.g., the absorption differences, the phase shift difference and the small angle scattering differences) and then modifies the baseline interference pattern 901 based on the aggregated differences for each detector pixel to generate the final interference pattern for the virtual object 601.

As noted above, in accordance with the disclosed projection-based approach, the projector component 112 determines the changes to the baseline interference pattern 801 for each of the sub-objects (i.e., the absorption object 702, the phase object 704 and the small angle scattering object 706) and each detector pixel as a function of direct paths of respective X-ray photons through the different sub-objects and without calculating deviated paths of the respective X-ray photons attributed to deflection.

In association with determining the absorption changes to the baseline interference pattern 901 caused by the absorption object, the phase shift changes caused by the phase object 704 and the small angle scattering changes caused by the small angle scattering object, the decomposition component 110 separately models the absorption object, the phase object and the small angle scattering objects as voxelized objects. With regard to the absorption object 702, the projector component 112 determines the absorption signals received at the respective detector elements based on projection of X-rays from the X-ray source 202 to the detector through the gratings and the voxelized absorption object following direct paths of the respective X-rays from the source through the object to the detector elements. The projector component 112 determines the absorption signals received at the respective detector elements based on the absorption profile (e.g., attenuation coefficient) of each voxel of the voxelized absorption object through which the respective X-rays pass along their direct path. In this regard, following a direct path of the X-ray beams from the X-ray source 202 through the voxelized absorption object to the respective detector elements, the projector component 112 determines, based on the absorption profiles of respective voxels through which respective X-ray beams pass, the amount decrease in amplitude β of the respective X-ray beams signals received at each of the detector elements in accordance with Equations 5-9. The projector component 112 integrates the absorption changes along the entire length of the X-ray path from the X-ray source 202 to the detector.

$$β = \frac{{n}_{a} {r}_{e} {λ}^{2}}{2 π} {f}_{2} , \qquad Equation 5$$

$$δ \frac{{n}_{a} {r}_{e} {λ}^{2}}{2 π} {f}_{1} , \qquad Equation 6$$

$$n = 1 - δ + i β , \qquad Equation 7$$

$${n}_{a} = \frac{ρ {N}_{a}}{{M}_{a}} , \qquad Equation 8$$

$$λ = \frac{h c}{E} , \qquad Equation 9$$

wherein:
β is the change in X-ray signal amplitude;
δ is the refraction amount (change of x-ray direction);
n is the refractive index;
λ is the X-ray signal wavelength;
f₁, f₂ are atomic scattering factors;
nₐ is the number density;
p is the physical density;
Mₐ is the molar mass
E is the X-ray energy
rₑ is the classical electron radius (rₑ=2.818·10⁻¹⁵ m)
Nₐ is the Avogadro constant (Nₐ=6.022·10²³ mol⁻¹)
h is the Planck constant (h=6.626·10⁻³⁴ Js=4.136·10⁻¹⁵ eV·s); and
c is the speed of light (c=2.997·10⁸ m/s)

With respect to the phase object 704, the projector component 112 calculates how the phase object 704 displaces or shifts the baseline interference pattern 801 based on the absolute phase object. The projector component 112 then evaluates the gradient in the direction perpendicular to the grating lines. In particular, the decomposition component 110 models the phase object 704 as a voxelized object composed of a plurality of voxels. The decomposition component 110 and/or the projector component 112 further calculates the refractive index n for each voxel in accordance with Equations 5-9. The decomposition component 110 and/or the projector component 112 further calculates the “refractive index gradient object” for the phase object based on the refractive indexes for each voxel in 3D space as aligned relative to the detector. In the general case, the refractive index gradient is a vector in 3D space. In cases wherein the grating lamellae are parallel to the z-axis, the vector can be reduced to a 2D vector (in the x-y-plane), as such an interferometer is insensitive to the gradient in z.

For example, in a voxelized virtual phase object, the projector component 112 can attribute a refractive index n to each voxel (i.e., wherein n=1-δ+iβ, Equation 7). For the phase object, we are only interested in the real part of the refractive index, (i.e., we are not calculating the absorption). Therefore, the refractive index can be simplified to Equation 10.

$$n = 1 - δ . \qquad Equation 10$$

The refractive index gradient object can then be calculated as follows in accordance with Equations 11 and 12.

$$∇ n = [ \begin{matrix}\frac{\partial n}{\partial x} \\ \frac{\partial n}{\partial y} \\ \frac{\partial n}{\partial z}\end{matrix} ] . \qquad Equation 11$$

Noting that for X-rays, & is very small, and therefore n≈1. Consequently:

$$Δ n = {n}_{2} - {n}_{1} \approx \frac{{n}_{2}}{{n}_{1}} - 1. \qquad Equation 12$$

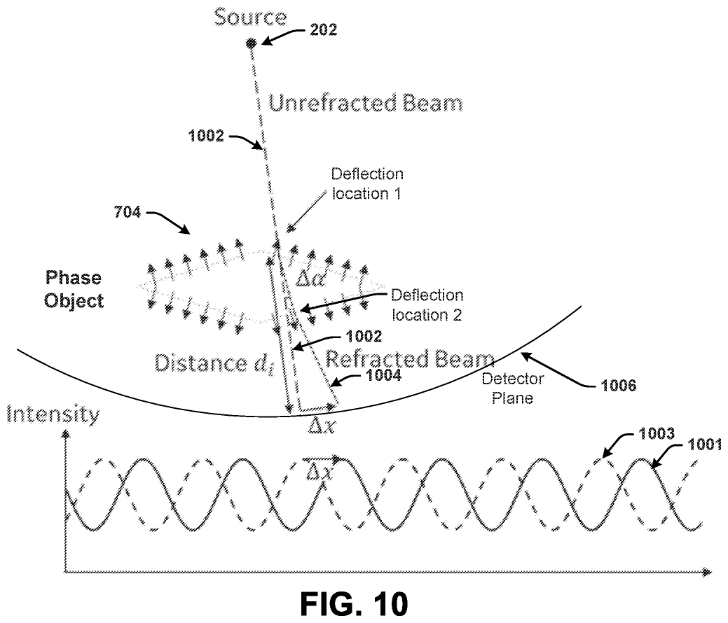

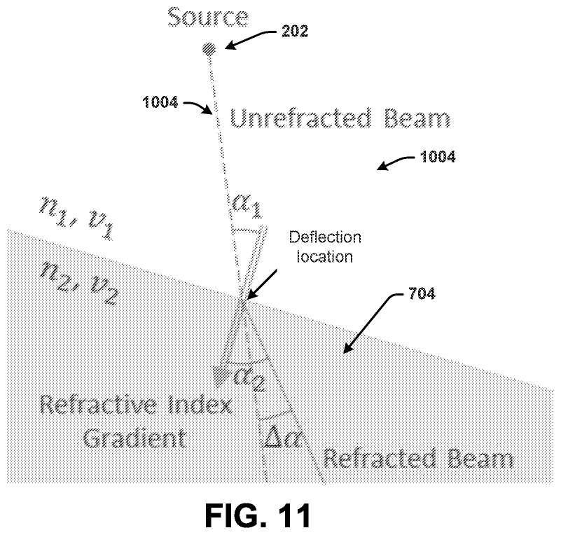

The projector component 112 further projects the source X-ray beams through the refractive index gradient object, calculating the sum of the products of the deflection angle and distance to the detector, as further illustrated with reference to FIGS. 10 and 11.

FIG. 10 illustrates aspects of the phase object projection simulation processing techniques in accordance with one or more embodiments of the disclosed subject matter. FIG. 10 illustrates projection of an X-ray beam from the X-ray source 202 through the phase object 704 toward the detector plane 1006 (e.g., wherein the detector plane comprises the X-ray detector 212 consisting of a plurality of detector elements 2121-k respectively corresponding to pixels). Line 1002 corresponds to the direct path of the X-ray beam (i.e., the unrefracted beam) through the phase object 704 and line 1004 corresponds to the actual path of the X-ray beam (i.e., the refracted beam). It should be appreciated that a single X-ray beam is illustrated for case of explanation and that in practice a plurality of X-rays are emitted from the X-ray source 202 through the phase object 704 to the detector. Dashed line 1003 indicates the baseline interference pattern received at the detector (not shown) and solid line 1001 indicates the phase object imparted interference pattern received at the detector.

The X-ray beam's direction will be straight in a homogeneous medium, (i.e., a medium with a constant refractive index). It only changes direction due to the gradient of the refractive index. The direction of the gradient is important. The beam does not change direction when the gradient is in the direction of the beam (such as for normal incidence on a surface). In the example shown, the actual path of the X-ray beam is assumed to correspond to the refracted beam (line 1004) which results from deflection by the object at a first deflection location, as the direction of the gradient of the refractive index at the second deflection location is assumed to be in the direction of the beam). The distance di between the location of the beam deflection (deflection location 1 in this example) and the detector, referred to herein as the “deflection distance,” is also important (unlike for absorption calculation). In this regard, the larger the deflection distance di between the deflection location and the detector, the larger the deflection amount Δx, at a given deflection angle Δa.

The change to the baseline interference pattern 1001 attributed to the phase object 704 is based on the deflection amount Δx to which the x-ray beam is deflected or refracted by phase object 704. For example, assume the x-ray beam passes from the X-ray source 202 through the phase object 704 and the beam is refracted at deflection location 1 by a given deflection angle Δa. The total deflection amount Δx (i.e., a distance) can be calculated in accordance with Equation 13, wherein dᵢ is the distance from the deflection location to the detector (i.e., the deflection distance). It should be appreciated that following the direct path of the unrefracted beam, through the phase object 704, there may be a plurality of different deflection locations (i) through the object depending on the modeled geometry and gradient index properties of the phase object. In association with determining the total deflection amount Δx for a given pixel, the projector component can aggregate individual deflection amounts respectively calculated for each deflection location and angle as weighted by the deflection distance.

$$Δ x = {\sum }_{i} ( {d}_{i} sin Δα ) . \qquad Equation 13$$

In some embodiments, because the deflection angle Δa is very small we can approximate sin Δa≈Δa and therefore Equation 13 can be reduced to Equation 14:

$$Δ x = {\sum }_{i} ( {d}_{i} \cdot Δ α ) . \qquad Equation 14$$

Because the total deflection Δx is generally much smaller than the pixel size, we can follow the path of the unrefracted beam, line 1002 (i.e., we do not need to calculate or determine the beam path for the refracted beam 1004). In this regard, as noted above, with the disclosed projection-based approach, we do not follow the deviated path or deflected path of the particle, but instead following the initial path and summing the deviations aggregated for each detector pixel as scaled based on the deflection distances (e.g., dᵢ). For example, as illustrated in FIG. 10, the changes to the baseline interference pattern 901 attributed to a phase object 704 include a shift to the baseline interference pattern by some amount (e.g., to the left or the right). For each detector pixel, the projector component 112 determines the shift distance (e.g., amount and direction) based on aggregation of estimated deflection amounts (Δx) of the X-ray photons relative to their direct paths (and corresponding direct pixel trajectory) attributed to the phase object weighted by the respective deflection distances.

Note that this assumes a system geometry where G₁ is in front of the object, and consequently sensitivity scales linearly with the distance from the detector. This is the system geometry that would typically be used in a phase-contrast CT system. In this regard, in association with calculating the deflection amount Δx for a given x-ray beam projected along its direct (unrefracted) path to the detector, the projector component 112 weighs the deflection amount Δa by the distance dᵢ between the voxel at the deflection location and the detector, and the same refraction angle will lead to a larger shift of the interference pattern as the distance between the voxel and the detector increases.

FIG. 11 illustrates calculating the deflection angle based on the refractive index gradient for the phase object 704. The deflection angle Δa of the refracted beam can be determined based on the refractive index gradient in accordance with Equations 15-18, wherein index nᵢ corresponds to a given refractive index, and vᵢ corresponds to a given phase velocity of the beam. The deflection angle is calculated for each voxel of the object along the X-ray beam path based on the two different refractive indices (n₁ and n₂) associated with each voxel, and more generically with the volumetric distribution of refractive indices in the vicinity of the pixel, from which the direction and magnitude of the refractive index gradient can be calculated. In particular, the deflection angle Δa of the refracted beam can be determined based on a first refractive index n₁ associated with the voxel (e.g., the refractive index of the medium between the object surface and the source (e.g., air) for the voxel located at the object surface and for all others, the refractive index at the beam entry location) the phase velocity v₁ of the beam prior to reaching the beam entry location (e.g., the surface of the object for the initial voxel located at the object surface) relative to the refractive index n₂ of the voxel of the object at the deflection location and the phase velocity v₂ of the beam at the deflection location using Equations 15-18.

$$Equation 15$$

$${v}_{i} = \frac{c}{{n}_{i}} .$$

$$Equation 16 ( Snell ’ s Law )$$

$$\frac{sin {α}_{1}}{sin {α}_{2}} = \frac{{n}_{2}}{{n}_{1}} .$$

Consequently, the angle a₂ of the refracted beam is given by:

$${α}_{2} = arcsin ( \frac{{n}_{1}}{{n}_{2}} sin {α}_{1} ) , \qquad Equation 17$$

and the angle Δa by which the beam is deflected is given by:

$$Δ α = {α}_{2} - {α}_{1} . \qquad Equation 18$$

Note that in grating-based X-ray phase contrast CT, the grating lamellae are generally parallel to the z-axis, i.e., the interferometer is insensitive to refraction in z. In this special case, we only need to calculate refraction in the x-y-plane.

With reference again to FIG. 9, with respect to the small angle scattering object 706, the projector component 112 calculates how the small angle scattering object changes the baseline interference pattern 901 based again on a voxelized representation of the small angel scatter object 706 and assuming a direct path of the X-ray particle. In this regard, as illustrated in FIG. 9 with respect to the small angle scattering object 706, the change between the baseline interference pattern 901 and the interference pattern caused by the small angle scattering object comprises a decrease in visibility and/or change in shape of the baseline interference pattern 901. The projector component 112 can determine these changes to the baseline interference pattern 901 attributed to the small angle scattering object 706 based on aggregation of estimated amounts of decrease in visibility of the X-ray photons relative to the direct paths attributed to the small angle scattering object weighted by the respective deflection distances. In association with determining the amounts of decrease in visibility of the X-ray photons for the small angle scattering object, instead of attributing an angle of deflection to each voxel, we attribute a standard deviation of scatter to each voxel, wherein the standard deviation of scatter is calculated based on the size of the particles, the transverse coherence length, the wavelength of the X-ray beam and the gratings period.

In this regard, the decomposition component 110 can model the small angle scattering object 706 as a phase contrast object in which the characteristic dimension (i.e., “particle” size, e.g., 1 μm to 10 μm) is much smaller than the detector resolution (e.g., 100 μm to 1 mm). In a voxelized representation of the small angle scattering object (e.g., a virtual object), the decomposition component 110 can attribute a scatter angle standard deviation σₐ to each voxel. Note that the mean of the scatter angle has already been accounted for in the phase object; consequently, for the small angle scattering object, the mean of the scatter angle distribution is zero.

The scatter angle standard deviation σₐ depends on both the magnitude of the refractive index variations and on the characteristic size of these variations with respect to the transverse coherence length of the X-ray beam (e.g., d of Equation 4). In some embodiments, the scatter angle standard deviation σₐ can be determined based on the transverse coherence length (an imaging system parameter that can be modeled and varied for different system configurations) and predetermined look up tables for different tissues. Additionally, or alternatively, the scatter angle standard deviation σₐ may be calculated from the measured linear diffusion coefficient ϵ.

In this regard, we assume the statistics of the scatter angle at any point to be a Gaussian random process with standard deviation σₐ. This is a reasonable assumption, particularly when utilizing the projection approximation in which the total scatter angle standard deviation is determined by the sum of many independent random scatter angle contributions along the beam path. The linear diffusion coefficient ϵ can be expressed by normalizing the scatter angle standard deviation σₐ to the sample thickness Δz in accordance with Equation 19.

$$ϵ = \frac{{σ}_{α}^{2}}{Δ z} . \qquad Equation 19$$

Similar to the process of calculating total beam deflection for the phase object, the projector component 112 applies a weighting to the diffusion coefficient ϵ as a function of the deflection distance dᵢ. If the beam were a plane wave, the scaling would be linear with dᵢ. However, in a cone beam geometry (which applies to all medical systems using X-ray tubes), the change of the correlation distance ξ also needs to be taken into account.

In the general case, a blur kernel can be applied to the fringe pattern. In the special case where the fringe pattern has been decomposed into sine waves, the computational burden can be reduced by simply calculating new amplitudes of the sine waves. This is due to the fact that a convolution is a linear operation, and as such it does not change the shape of a sine wave, but only changes its amplitude.

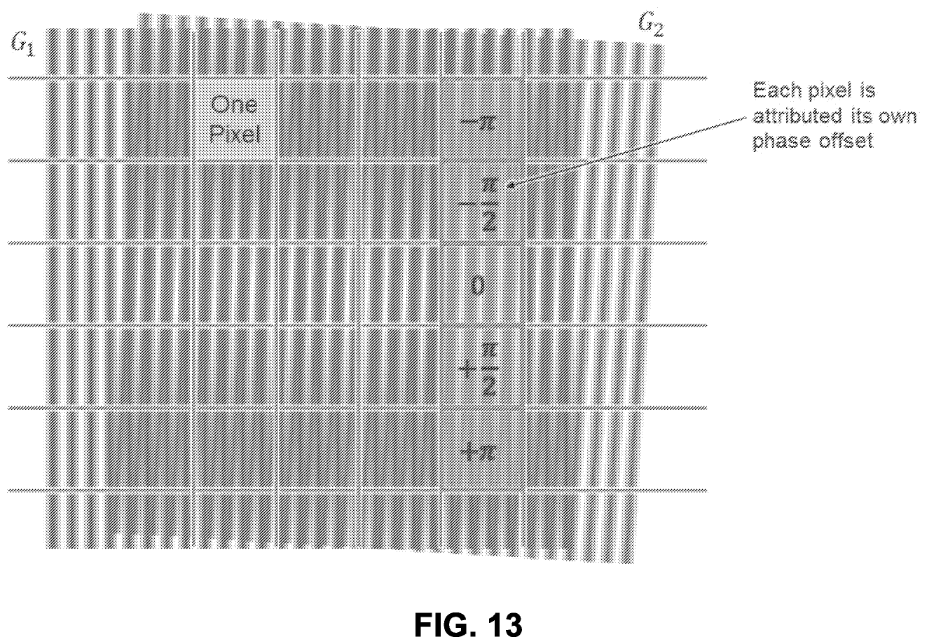

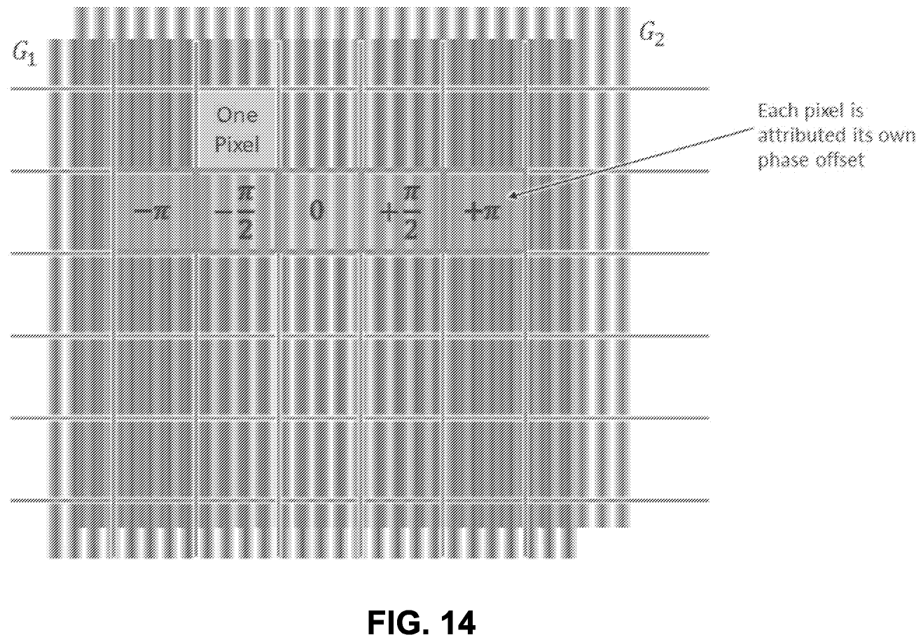

With reference again to FIG. 6, as discussed above, prior to projection simulation through the respective decomposed sub-objects, the source component 106 models the spot profile as multiple point sources (at 602) and the baseline component 108 calculates the baseline interference pattern at 604. The different point sources will all see essentially the same grating tilt angles, but they may be seeing different effective grating offsets, as described in greater detail infra with reference to FIGS. 13 and 14. In various embodiments, the alignment of the gratings G₀, G₁ and/or G₂ with respect to the X-ray beam direction may vary. For example, different system parameters may result in a different tilt of the (typically high-aspect-ratio) grating G₀ around its grating lamellae. Variations in grating alignment can result in changes to the X-ray modulation waveform. These variations may be due to difficulties in alignment, as well as due to mechanical deformations (e.g., the CT gantry slightly deforming at high gantry speeds). Gratings may also be tilted on purpose around the axis of the grating lamellae for optimizing the grating absorption profile, such as for increasing the contrast (i.e., the amplitude/offset) of the system. Thus, in various embodiments, the baseline component 108 can account for different grating alignments in association with modeling the baseline interference pattern.

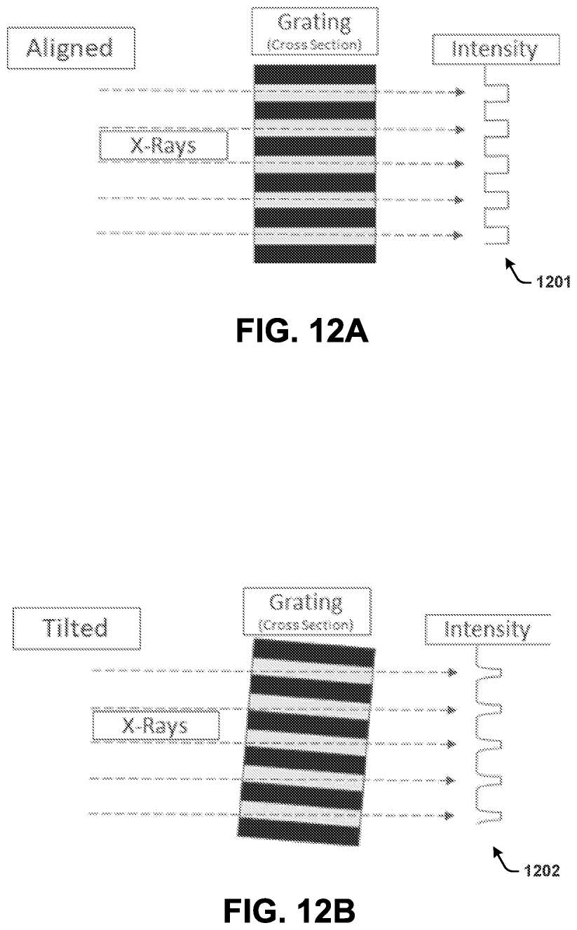

FIGS. 12A and 12B illustrate influence of aligned and tilted gratings on the X-ray beams of a grating-based X-ray phase contrast imaging system. FIG. 12A presents a cross-sectional view of an aligned grating and FIG. 12B presents a cross-sectional view of a tilted grating. As illustrated in comparison of the interference pattern 1201 and interference pattern 1202, a grating tilt around the direction of the grating lamellae changes the X-ray modulation waveform, for both the absorption grating G₀ and the phase grating G₁. This effect needs to be incorporated in the simulation of the baseline signal when applicable, such as for different spot profiles and design purposes. When the gratings are slightly tilted or have slightly unmatched periods, different point sources will “see” different phase offsets. When these different phase offsets are added, the overall contrast of the interference pattern will be reduced. To this end, the baseline component 108 can model two misaligned gratings as an offset displacement between the two gratings in each of the corresponding detector pixels and apply the offset to the baseline interference pattern at the corresponding detector pixels, as illustrated in FIGS. 13 and 14. In this regard, FIG. 13 illustrates example phase offsets for gratings tilted in their plane. Each pixel is attributed is own phase offset determined by the baseline component 108. FIG. 14 illustrates example phase offsets for gratings with unmatched periods. In a cone beam geometry, grating displacement in the direction of the beam changes the magnification factor. This changes the apparent period of the grating (as seen from the detector), thereby leading to unmatched grating periods.

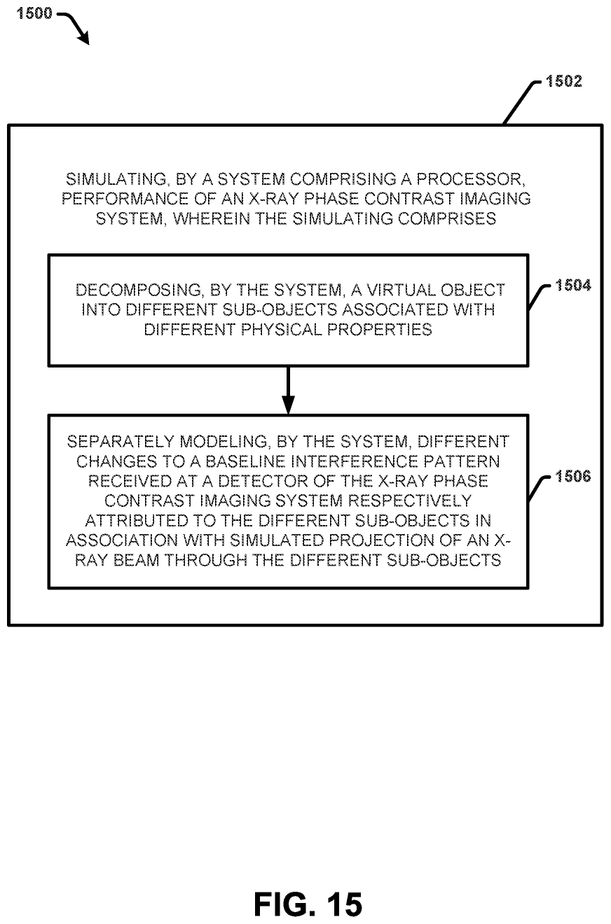

FIG. 15 presents a flow diagram of an example computer-implemented method 1500 for simulating performance of a grating-based X-ray phase contrast imaging system in accordance with one or more embodiments of the disclosed subject matter. In accordance with method 1500, at 1502 a system comprising a processor (e.g., system 100), simulates performance of an X-ray phase contrast imaging system, wherein the simulating comprises decomposing, by the system, a virtual object into different sub-objects associated with different physical properties at 1504 and at 1506, separately modeling, by the system, different changes to a baseline interference pattern received at a detector of the X-ray phase contrast imaging system respectively attributed to the different sub-objects in association with simulated projection of an X-ray beam through the different sub-objects.

In various embodiments, the different sub-objects comprise an absorption object, a phase object and a small angle scattering object, and wherein the different changes respectively correspond to an absorption change, a phase shift change, and a dark field change. The detector of method 1500 comprises a plurality of pixels and wherein the separately modeling comprises separately modeling the different changes for each pixel of the plurality of pixels. In accordance with method 1500, the separately modeling comprises determining the changes as a function of direct paths of respective X-ray photons through the different sub-objects and without calculating deviated paths of the respective X-ray photons attributed to deflection. As applied to the phase object, the changes comprise a shift to the baseline interference pattern attributed to the phase object, and wherein the separately modeling comprises determining the shift based on aggregation of estimated deflections amounts of the X-ray photons relative to the direct paths attributed to the phase object weighted by respective deflection distances of the X-ray photons. As applied to the small angle scattering object, the changes comprise a decrease in visibility to the baseline interference pattern attributed to the small angle scattering object, and wherein the separately modeling comprises determining the decrease in visibility based on aggregation of estimated amounts of decrease in visibility of the X-ray photons relative to the direct paths attributed to the small angle scattering object weighted by respective deflection distances of the X-ray photons.

In some embodiments, method 1500 may further comprise combining, by the system, the different changes together to generate an aggregated interference pattern for the virtual object, and generating, by the system, different images based on the aggregated interference pattern and defined configuration parameters of the X-ray phase contrast imaging system using one or more image generation models.

Additionally, or alternatively, method 1500 may further comprise, determining, by the system, a preferred improvement to one or more characteristics of the different images and adjusting, by the system, one or more of the defined configuration parameters to achieve the preferred improvement. Still in other embodiments, method 1500 may further comprise determining, by the system, a preferred improvement to one or more characteristics of the different images, and adjusting, by the system, one or more parameters of the one or more image generation models to achieve the preferred improvement.

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, procedural programming languages, such as the “C” programming language or similar programming languages, and machine-learning programming languages such as like CUDA, Python, Tensorflow, PyTorch, and the like. The computer readable program instructions can execute entirely on the user's computer, partly on the user's computer, as a stand-alone software package, partly on the user's computer and partly on a remote computer or entirely on the remote computer or server using suitable processing hardware. In the latter scenario, the remote computer can be connected to the user's computer through any type of network, including a local area network (LAN) or a wide area network (WAN), or the connection can be made to an external computer (for example, through the Internet using an Internet Service Provider). In various embodiments involving machine-learning programming instructions, the processing hardware can include one or more graphics processing units (GPUs), central processing units (CPUs), and the like. For example, one or more of the disclosed machine-learning models (e.g., the image transformation model 908, the deep learning network 1010 and/or combinations thereof) may be written in a suitable machine-learning programming language and executed via one or more GPUs, CPUs or combinations thereof. In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

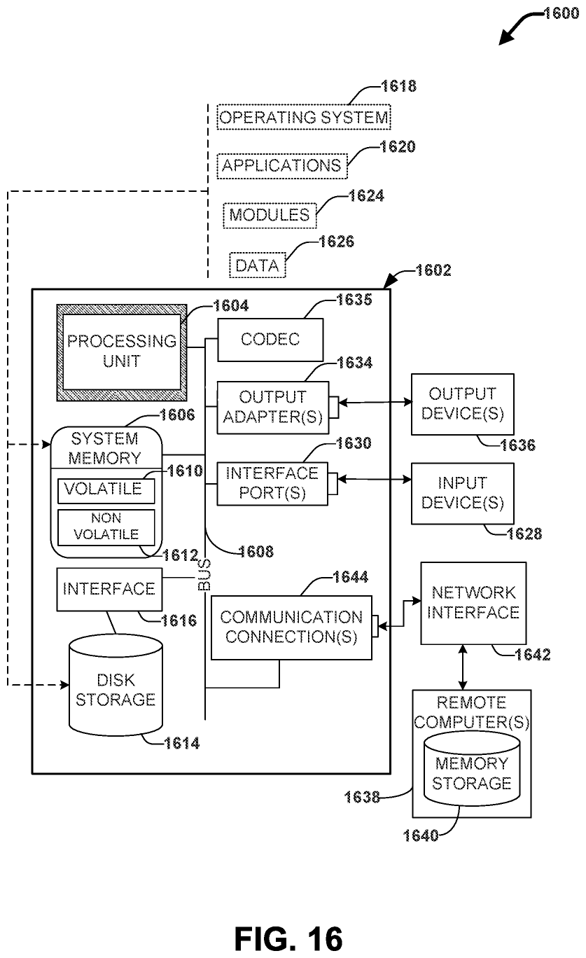

In connection with FIG. 16, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 16, an example environment 1600 for implementing various aspects of the claimed subject matter includes a computer 1602. The computer 1602 includes a processing unit 1604, a system memory 1606, a codec 1635, and a system bus 1608. The system bus 1608 couples system components including, but not limited to, the system memory 1606 to the processing unit 1604. The processing unit 1604 can be any of various available processors. Dual microprocessors, one or more GPUs, CPUs, and other multiprocessor architectures also can be employed as the processing unit 1604.

The system bus 1608 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1694), and Small Computer Systems Interface (SCSI).

The system memory 1606 includes volatile memory 1610 and non-volatile memory 1612, which can employ one or more of the disclosed memory architectures, in various embodiments. The basic input/output system (BIOS), containing the basic routines to transfer information between elements within the computer 1602, such as during start-up, is stored in non-volatile memory 1612. In addition, according to present innovations, codec 1635 can include at least one of an encoder or decoder, wherein the at least one of an encoder or decoder can consist of hardware, software, or a combination of hardware and software. Although, codec 1635 is depicted as a separate component, codec 1635 can be contained within non-volatile memory 1612. By way of illustration, and not limitation, non-volatile memory 1612 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1612 can employ one or more of the disclosed memory devices, in at least some embodiments. Moreover, non-volatile memory 1612 can be computer memory (e.g., physically integrated with computer 1602 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1610 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1602 can also include removable/non-removable, volatile/non-volatile computer storage medium. FIG. 16 illustrates, for example, disk storage 1614. Disk storage 1614 includes, but is not limited to, devices like a magnetic disk drive, solid state disk (SSD), flash memory card, or memory stick. In addition, disk storage 1614 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1614 to the system bus 1608, a removable or non-removable interface is typically used, such as interface 1616. It is appreciated that disk storage 1614 can store information related to a user. Such information might be stored at or provided to a server or to an application running on a user device. In one embodiment, the user can be notified (e.g., by way of output device(s) 1636) of the types of information that are stored to disk storage 1614 or transmitted to the server or application. The user can be provided the opportunity to opt-in or opt-out of having such information collected or shared with the server or application (e.g., by way of input from input device(s) 1628).

It is to be appreciated that FIG. 16 describes software that acts as an intermediary between users and the basic computer resources described in the suitable operating environment 1600. Such software includes an operating system 1618. Operating system 1618, which can be stored on disk storage 1614, acts to control and allocate resources of the computer 1602.

Applications 1620 take advantage of the management of resources by operating system 1618 through program modules 1624, and program data 1626, such as the boot/shutdown transaction table and the like, stored either in system memory 1606 or on disk storage 1614. It is to be appreciated that the claimed subject matter can be implemented with various operating systems or combinations of operating systems.

A user enters commands or information into the computer 1602 through input device(s) 1628. Input devices 1628 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1604 through the system bus 1608 via interface port(s) 1630. Interface port(s) 1630 include, for example, a serial port, a parallel port, a game port, and a universal serial bus (USB). Output device(s) 1636 use some of the same type of ports as input device(s) 1628. Thus, for example, a USB port can be used to provide input to computer 1602 and to output information from computer 1602 to an output device 1636. Output adapter 1634 is provided to illustrate that there are some output devices 1636 like monitors, speakers, and printers, among other output devices 1636, which require special adapters. The output adapters 1634 include, by way of illustration and not limitation, video and sound cards that provide a means of connection between the output device 1636 and the system bus 1608. It should be noted that other devices or systems of devices provide both input and output capabilities such as remote computer(s) 1638.

Computer 1602 can operate in a networked environment using logical connections to one or more remote computers, such as remote computer(s) 1638. The remote computer(s) 1638 can be a personal computer, a server, a router, a network PC, a workstation, a microprocessor based appliance, a peer device, a smart phone, a tablet, or other network node, and typically includes many of the elements described relative to computer 1602. For purposes of brevity, only a memory storage device 1640 is illustrated with remote computer(s) 1638. Remote computer(s) 1638 is logically connected to computer 1602 through a network interface 1642 and then connected via communication connection(s) 1644. Network interface 1642 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1644 refers to the hardware/software employed to connect the network interface 1642 to the bus 1608. While communication connection 1644 is shown for illustrative clarity inside computer 1602, it can also be external to computer 1602. The hardware/software necessary for connection to the network interface 1642 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store.” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer-executable components; and
a processor that executes the computer-executable components stored in the memory, wherein the computer-executable components comprise:
a simulation component that simulates performance of an X-ray phase contrast imaging system using a simulation model, the simulation model comprising:
a decomposition component that decomposes a virtual object into different sub-objects associated with different physical properties; and
a projector component that separately models different changes to a baseline interference pattern received at a detector of the X-ray phase contrast imaging system respectively attributed to the different sub-objects in association with simulated projection of an X-ray beam through the different sub-objects.

2. The system of claim 1, wherein the different sub-objects comprise an absorption object, a phase object and a small angle scattering object.

3. The system of claim 1, wherein the different changes respectively correspond to an absorption change, a phase change, and a dark field change.

4. The system of claim 1, wherein the detector comprises a plurality of pixels and wherein the projector component separately models the different changes for each pixel of the plurality of pixels.

5. The system of claim 4, wherein the projector component determines the changes as a function of direct paths of respective X-ray photons through the different sub-objects and without calculating deviated paths of the respective X-ray photons attributed to deflection.

6. The system of claim 5, wherein the different sub-objects comprise a phase object, wherein the changes comprise a shift to the baseline interference pattern attributed to the phase object, and wherein the projector component determines the shift based on aggregation of estimated deflection amounts of the X-ray photons relative to the direct paths attributed to the phase object weighted by respective deflection distances of the X-ray photons.

7. The system of claim 5, wherein the different sub-objects comprise a small angle scattering object, wherein the changes comprise a decrease in visibility to the baseline interference pattern attributed to the small angle scattering object, and wherein the projector component determines the decrease in visibility based on aggregation of estimated amounts of decrease in visibility of the X-ray photons relative to the direct paths attributed to the small angle scattering object weighted by respective deflection distances of the X-ray photons.

8. The system of claim 1, wherein the simulation model further comprises:
a detector component that combines the different changes together to generate an aggregated interference pattern for the virtual object.

9. The system of claim 1, wherein the computer-executable components further comprise:
an image generation component that generates different images based on the different changes and defined configuration parameters of the X-ray phase contrast imaging system using one or more image generation models.

10. The system of claim 9, wherein the computer-executable components further comprise:
an optimization component that determines a preferred improvement to one or more characteristics of the different images and optimizes one or more of the defined configuration parameters to achieve the preferred improvement.

11. The system of claim 9, wherein the computer-executable components further comprise:
an optimization component that determines a preferred improvement to one or more characteristics of the different images and optimizes one or more parameters of the one or more image generation models to achieve the preferred improvement.

12. The system of claim 1, wherein the X-ray phase contrast imaging system comprises a computed tomography imaging system with phase contrast functionality and wherein the performance corresponds to performance of a computed tomography scan.

13. A method, comprising:
simulating, by a system comprising a processor, performance of an X-ray phase contrast imaging system, wherein the simulating comprises:
decomposing, by the system, a virtual object into different sub-objects associated with different physical properties; and
separately modeling, by the system, different changes to a baseline interference pattern received at a detector of the X-ray phase contrast imaging system respectively attributed to the different sub-objects in association with simulated projection of an X-ray beam through the different sub-objects.

14. The method of claim 13, wherein the different sub-objects comprise an absorption object, a phase object and a small angle scattering object, and wherein the different changes respectively correspond to an absorption change, a phase change, and a dark field change.

15. The method of claim 13, wherein the detector comprises a plurality of pixels and wherein the separately modeling comprises separately modeling the different changes for each pixel of the plurality of pixels.

16. The method of claim 15, wherein the separately modeling comprises determining the changes as a function of direct paths of respective X-ray photons through the different sub-objects and without calculating deviated paths of the respective X-ray photons attributed to deflection.

17. The method of claim 16, wherein the different sub-objects comprise a phase object, wherein the changes comprise a shift to the baseline interference pattern attributed to the phase object, and wherein the separately modeling comprises determining the shift based on aggregation of estimated deflection amounts of the X-ray photons relative to the direct paths attributed to the phase object weighted by respective deflection distances of the X-ray photons.

18. The method of claim 16, wherein the different sub-objects comprise a small angle scattering object, wherein the changes comprise a decrease in visibility to the baseline interference pattern attributed to the small angle scattering object, and wherein the separately modeling comprises determining the decrease in visibility based on aggregation of estimated amounts of decrease in visibility of the X-ray photons relative to the direct paths attributed to the small angle scattering object weighted by respective deflection distances of the X-ray photons.

19. The method of claim 13, further comprising:
combining, by the system, the different changes together to generate an aggregated interference pattern for the virtual object; and
generating, by the system, different images based on the aggregated interference pattern and defined configuration parameters of the X-ray phase contrast imaging system using one or more image generation models.

20. A non-transitory machine-readable storage medium, comprising executable instructions that, when executed by a processor, facilitate performance of operations, comprising:
simulating performance of an X-ray phase contrast imaging system, wherein the simulating comprises:
decomposing a virtual object into different sub-objects associated with different physical properties; and
separately modeling different changes to a baseline interference pattern received at a detector of the X-ray phase contrast imaging system respectively attributed to the different sub-objects in association with simulated projection of an X-ray beam through the different sub-objects.
